(19) United States
(12) Patent Application Publication
(10) Pub. No.: US 2019/0200864 A1
(43) Pub. Date: Jul. 4, 2019

(54) Mobile Application for Use With Young Patients Who Have A Chronic Illness

(71) Applicant: The California State University - San Marcos, San Marcos, CA (US)

(72) Inventors: Youwen Ouyang, San Marcos, CA (US); Pamela Kohlbry, San Marcos, CA (US)

(73) Assignee: The California State University - San Marcos, San Marcos, CA (US)

(21) Appl. No.: 16/295,010

(22) Filed: Mar. 7, 2019

Related U.S. Application Data
(63) Continuation-in-part of application No. 14/918,305, filed on Oct. 20, 2015.

(51) Int. Cl. A61B 5/00; G06F 21/60; G06F 21/62; H04W 4/021

(52) U.S. Cl. CPC A61B 5/0022; G06F 21/602; H04W 68/00; H04W 4/021; A61B 5/747; G06F 21/6245

(57) ABSTRACT

Methods for providing a chronically ill patient with medical alerts, pharmacological alerts and information are disclosed that include: providing a patient with a chronic illness; providing a mobile device comprising a GPS receiver, a display, a microprocessor and a wireless communication transceiver, the mobile device programmed to process encrypted medical data, medical tags and medical information through an application; providing a server comprising a central processing unit, a memory, a clock and a server communication transceiver that receives input from the wireless communication transceiver of the mobile device, the memory having encrypted medical data, medical tags and medical information stored therein for a plurality of chronically ill patients, the central processing unit programmed to implement the following method comprising: receiving encrypted medical data, medical tags and medical information from the mobile device; analyzing the encrypted medical data, medical tags and medical information from the mobile device; generating a new set of unique and revised encrypted medical data, medical tags and medical information; providing the new set of unique and revised encrypted medical data, medical tags and medical information to the mobile device, wherein the mobile device updates the application with the unique and revised encrypted medical data, medical tags and medical information, which is then accessible by a user of the mobile device.

This Continuation in Part Application claims priority to U.S. Utility application Ser. No. 14/918,305 filed on Oct. 20, 2015, which claims priority to U.S. Provisional Application Ser. No. 62/066,535 filed on Oct. 21, 2014 and entitled “Mobile Application For Use With Young Patients Who Have A Chronic Illness”, all of which are commonly-owned and incorporated herein in their entirety by reference.

### FIELD OF THE SUBJECT MATTER

The field of the subject matter is a mobile application for use with patients, especially younger patients, such as children, pre-teenagers, teenagers and young adults, who have a chronic illness.

### BACKGROUND

Chronic conditions require patients to manage their care over a long period of time. Research exists that patient self-management skills are crucial to their understanding and involvement with their health (Clark and Paraska, 2013). There are a number of ways to handle this type of patient self-management from providing written information that is sent home with the patient and/or patient's caregivers/family, providing nurse and nurse practitioner hotlines for the patient to call, and providing walk-in clinic information, if the patient isn't physically located near his or her doctor.

Clearly, however, there are significant needs for patients with chronic illnesses who are not being addressed by current patient interaction systems or methods. For example, chronic conditions may require information that is updated daily or even hourly depending on the patient's current symptoms or condition. Chronic conditions may require that people are updated and prescriptions are requested and refilled without initiation by the patient. The patient may not live near his or her primary care physician or near a clinic, so that a “drop-in visit” is not convenient. The patient may travel domestically or internationally on a regular basis, such that normally he or she would need to map out clinics, secure doctors in advance and take various medications with him or her in order to be prepared for any need while traveling. The patient may not be able to carry written information with him or her all the time and may not be able to access it easily and reliably, especially if there are more than 5-10 pages of information for the condition. Therefore, there are needs by patients with chronic illnesses that are not being addressed or able to be addressed by conventional methods and protocols.

The term mHealth refers to the virtual world of “mobile computing, medical sensor, and communications technologies” that expands healthcare for chronic disease beyond the healthcare provider's office (Eng and Lee, 2013). Healthcare education as it has existed wasn't designed to teach or interact with today's technology savvy young patients. In comparison to 10 years ago, there has been an exponential increase in the number of children using cell phones. Roughly 78% of teenager's ages 12-17 now have cellphones, and half of those are smartphones (Madden, et al, 2013).

Children, as young as 2 years old navigate their own potty training with “The Pull Ups Big Kid App” by Huggies®. It is imperative that healthcare embrace mobile technologies to remain “plugged” into patients' needs, along with addressing significant needs in the area of patient care. Findings from the Institute for Healthcare Informatics 2013 report on 40,000 healthcare apps did not identify any software applications for children with chronic illnesses. Healthcare mobile applications or mobile apps designed for children with chronic illnesses have the potential to address a growing demographic of young healthcare recipients, while at the same time addressing most, if not all, of the problems described earlier with conventional patient care. Transitional care management from adolescence to adult is complex for patients and transition care givers (Kelly, 2014).

mHealth apps could encourage a child's understanding of their own health, perceived self-efficacy, and improve long-term management of their chronic condition. In addition, there is an opportunity with these applications to provide regular interactivity, including 24/7 interactivity in some embodiments, and patient care as the child gets older and transitions into being an adult. A brief Google search was conducted to determine what apps are currently available for children with chronic conditions (Table 1). Mobile apps for common illnesses such as diabetes were found. Websites that provide reviews of mobile apps for children with chronic illness were identified (Table 2). There was a lack of research on the use of health apps for children and for uncommon chronic illnesses, such as Thalassemia, where this kind of innovation would be necessary and useful.

Boucher (US Patent Publication 2014/0073880) which may be considered related art does not have or describe embodiments where contemplated systems can provide information on an on-going basis without prompting from the patient. This feature is exceptionally important for chronically ill patients, as discussed earlier. There are some conditions, including Thalassemia that require new and different modes of operation and methods of action.

Therefore, it would be ideal to develop an interactive and patient-specific application for a mobile device that allows a health practitioner and a patient to interact in a number of ways designed to make the patient experience more comprehensive and health-driven, including providing games and learning modules for children and young adults, where these transition as the child ages, the ability to find the patient and provide information about specific physicians, pharmacies and clinics in the patient's immediate area, track medication, set specific alarms for the patient's medication and adjust the medication, if the patient misses a dose, along with other useful interactive functionality.

### SUMMARY OF THE SUBJECT MATTER

Methods for providing a chronically ill patient with medical alerts, pharmacological alerts and information are disclosed that include: providing a patient with a chronic illness; providing a mobile device comprising a GPS receiver, a display, a microprocessor and a wireless communication transceiver, the mobile device programmed to process encrypted medical data, medical tags and medical information through an application; providing a server comprising a central processing unit, a memory, a clock and a server communication transceiver that receives input from the wireless communication transceiver of the mobile device, the memory having encrypted medical data, medical tags and medical information stored therein for a plurality of chronically ill patients, the central processing unit programmed to implement the following method comprising: receiving encrypted medical data, medical tags and medical information from the mobile device; analyzing the encrypted medical data, medical tags and medical information from the mobile device; generating a new set of unique and revised encrypted medical data, medical tags and medical information; providing the new set of unique and revised encrypted medical data, medical tags and medical information to the mobile device, wherein the mobile device updates the application with the unique and revised encrypted medical data, medical tags and medical information, which is then accessible by a user of the mobile device.

Methods for providing a chronically ill patient with medical alerts, pharmacological alerts and information are also disclosed that include: providing a mobile device comprising a GPS receiver, a display, a microprocessor and a wireless communication transceiver, the mobile device programmed to process encrypted medical data, medical tags and medical information through an application; providing a server comprising a central processing unit, a memory, a clock and a server communication transceiver that receives input from the wireless communication transceiver of the mobile device, the memory having encrypted medical data, medical tags and medical information stored therein for a plurality of chronically ill patients, the central processing unit programmed to the central processing unit programmed to implement the following method comprising: receiving encrypted medical data, medical tags and medical information from the mobile device; receiving GPS information from the wireless communication transceiver from the mobile device; analyzing the encrypted medical data, medical tags and medical information from the mobile device; determining location information for a medical facility, healthcare provider or pharmacy utilizing the GPS information from the wireless communication transceiver from the mobile device and the encrypted medical data, medical tags and medical information from the mobile device; providing the location information to the mobile device without prompting from the patient, wherein the mobile device notifies a user of the mobile device of this location information.

Methods for providing a chronically ill patient with medical alerts, pharmacological alerts and information are also disclosed that include: providing a patient with a chronic illness; providing a mobile device comprising a GPS receiver, a display, a microprocessor and a wireless communication transceiver, the mobile device programmed to process encrypted medical data, medical tags and medical information through an application; providing a server comprising a central processing unit, a memory, a clock and a server communication transceiver that receives input from the wireless communication transceiver of the mobile device, the memory having encrypted medical data, medical tags and medical information stored therein for a plurality of chronically ill patients, the central processing unit programmed to implement the following method comprising: receiving the patient's specific encrypted medical data, medical tags and medical information from the mobile device; analyzing the encrypted medical data, medical tags and medical information from the mobile device; generating a new set of the patient's unique and revised encrypted medical data, medical tags and medical information; providing the new set of unique and revised encrypted medical data, medical tags and medical information to the mobile device wherein providing the new set of unique and revised encrypted medical data, medical tags and medical information to the mobile device is initiated or completed without prompting from the patient.

### BRIEF DESCRIPTION OF THE FIGURES

FIG. 1 shows a contemplated screen shot on a mobile device for an app.

FIG. 2 shows a contemplated system diagram.

FIG. 3 shows a contemplated security component.

FIG. 4 shows several contemplated functions.

FIG. 5 shows a contemplated method of use.

### DETAILED DESCRIPTION

At California State University San Marcos (CSUSM) School of Nursing attention is being focused on nursing informatics which includes electronic medical records and mobile applications across the nursing curriculum. To meet this growing need Dr. Kohlbry, a nursing faculty, initiated an option for a class assignment for undergraduate nursing students in a health promotion class to work on a project to create the conceptual functionality and design of a mobile app for children with a chronic illness, such as the blood disorder, Thalassemia. The faculty and students discussed the needs of children, parents, and practitioners with Susan Carson, RN, MSN, CPNP, Lead Nurse Practitioner of the Thalassemia Program in the Hematology Program, at Children's

Hospital Los Angeles. Carson described one of the most important considerations for children with Thalassemia is the difficulty in the transition period from childhood to adulthood. Motivating, engaging, and educating children is the first step to overcoming potential challenges they may face. It is important to foster treatment adherence and establish support beyond parent's oversight, long before the transition period occurs.

As a result of this work, an interactive and patient-specific application has been conceptualized for a mobile device that allows a health practitioner and a patient to interact in a number of ways designed to make the patient experience more comprehensive and health-driven, including providing games and learning modules for children and young adults, where these transition as the child ages, the ability to find the patient and provide information about specific physicians, pharmacies and clinics in the patient's immediate area, track medication, set specific alarms for the patient's medication and adjust the medication, if the patient misses a dose, along with other useful interactive functionality. The ultimate goal of this disclosure is to provide information about systems and methods that improve adherence and quality of life by using contemplated embodiments and applications to build skills, so that when the teenage patient transitions to adult self-care, that patient knows their medication regime, can easily access resources, prescriptions, and practitioners, and can understand and utilize their own medical and health information.

Methods for providing a chronically ill patient with medical alerts, pharmacological alerts and information are disclosed that include: providing a patient with a chronic illness; providing a mobile device comprising a GPS receiver, a display, a microprocessor and a wireless communication transceiver, the mobile device programmed to process encrypted medical data, medical tags and medical information through an application; providing a server comprising a central processing unit, a memory, a clock and a server communication transceiver that receives input from the wireless communication transceiver of the mobile device, the memory having encrypted medical data, medical tags and medical information stored therein for a plurality of chronically ill patients, the central processing unit programmed to implement the following method comprising: receiving encrypted medical data, medical tags and medical information from the mobile device; analyzing the encrypted medical data, medical tags and medical information from the mobile device; generating a new set of unique and revised encrypted medical data, medical tags and medical information; providing the new set of unique and revised encrypted medical data, medical tags and medical information to the mobile device, wherein the mobile device updates the application with the unique and revised encrypted medical data, medical tags and medical information, which is then accessible by a user of the mobile device.

Methods for providing a chronically ill patient with medical alerts, pharmacological alerts and information are also disclosed that include: providing a mobile device comprising a GPS receiver, a display, a microprocessor and a wireless communication transceiver, the mobile device programmed to process encrypted medical data, medical tags and medical information through an application; providing a server comprising a central processing unit, a memory, a clock and a server communication transceiver that receives input from the wireless communication transceiver of the mobile device, the memory having encrypted medical data, medical tags and medical information stored therein for a plurality of chronically ill patients, the central processing unit programmed to the central processing unit programmed to implement the following method comprising: receiving encrypted medical data, medical tags and medical information from the mobile device; receiving GPS information from the wireless communication transceiver from the mobile device; analyzing the encrypted medical data, medical tags and medical information from the mobile device; determining location information for a medical facility, healthcare provider or pharmacy utilizing the GPS information from the wireless communication transceiver from the mobile device and the encrypted medical data, medical tags and medical information from the mobile device; providing the location information to the mobile device without prompting from the patient, wherein the mobile device notifies a user of the mobile device of this location information.

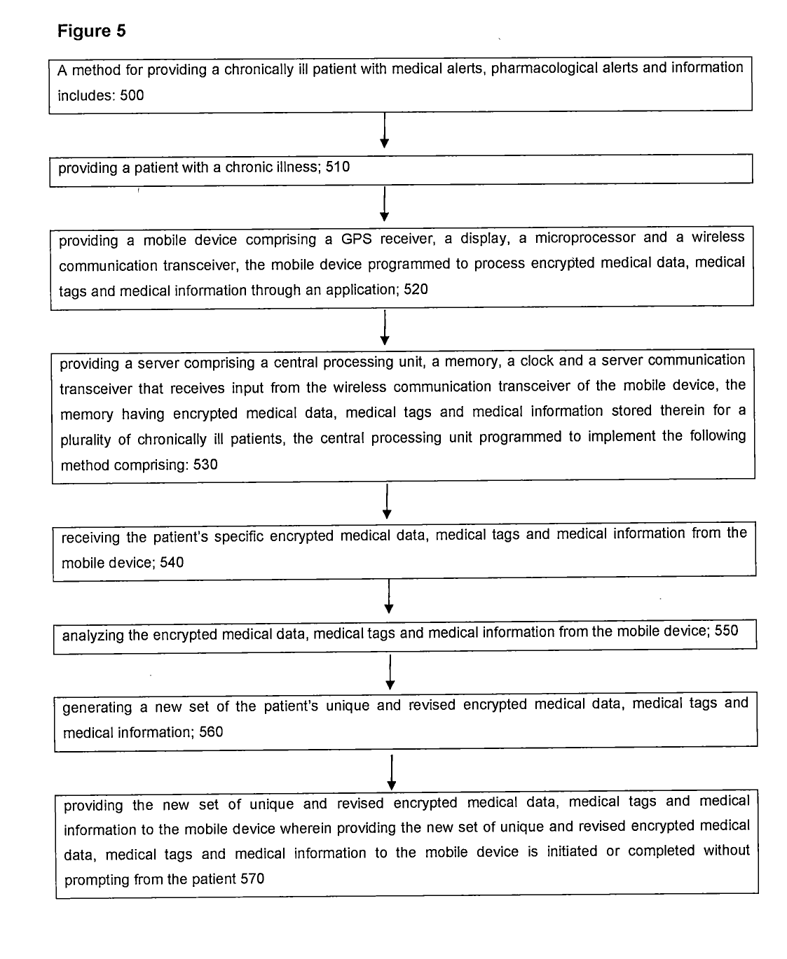

Methods for providing a chronically ill patient with medical alerts, pharmacological alerts and information are also disclosed that include 500: providing a patient with a chronic illness 510; providing a mobile device comprising a GPS receiver, a display, a microprocessor and a wireless communication transceiver, the mobile device programmed to process encrypted medical data, medical tags and medical information through an application 520; providing a server comprising a central processing unit, a memory, a clock and a server communication transceiver that receives input from the wireless communication transceiver of the mobile device, the memory having encrypted medical data, medical tags and medical information stored therein for a plurality of chronically ill patients, the central processing unit programmed to implement the following method comprising 530: receiving the patient's specific encrypted medical data, medical tags and medical information from the mobile device; 540 analyzing the encrypted medical data, medical tags and medical information from the mobile device 550; generating a new set of the patient's unique and revised encrypted medical data, medical tags and medical information 560; providing the new set of unique and revised encrypted medical data, medical tags and medical information to the mobile device wherein providing the new set of unique and revised encrypted medical data, medical tags and medical information to the mobile device is initiated or completed without prompting from the patient 570. This method is shown in FIG. 5.

As mentioned earlier and throughout this disclosure, the work conducted on the application and how it can be utilized made it immediately apparent that the contemplated application could be utilized for other chronic illnesses, because the overriding feature of chronic illness is that they do not heal or are cured quickly, they require constant monitoring, and it can be difficult to regularly and properly monitor and treat it for patients who are younger and/or not as mature as an adult. Thalassemia is one of those chronic illnesses and the chronic illness that initiated this work and is the focus of many of the contemplated embodiments. Therefore, contemplated applications are not merely abstract ideas that are being implemented by a computer, but instead have practical applications that require significantly more in the way of data and performance models.

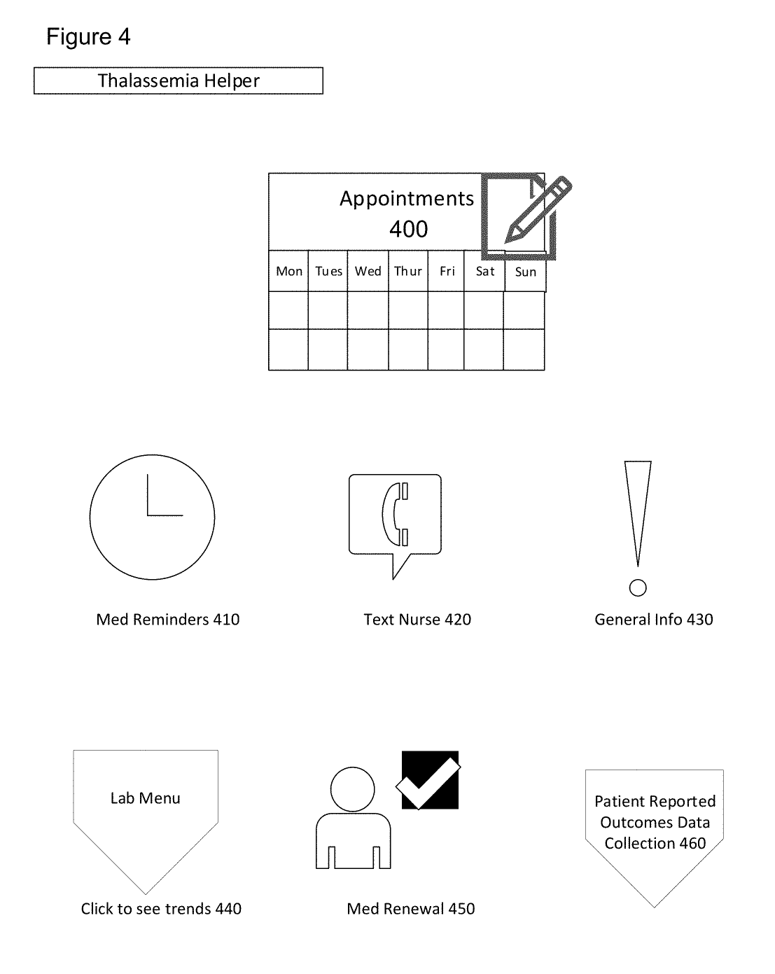

With respect to functionality of systems and methods disclosed herein, there are several contemplated functions that are important to consider as part of this disclosure. Several of these functions are shown in FIG. 4. The appointment function is shown as part of this figure under 400. Additional functions are medicine reminders 410, texting the nurse or medical practitioner 420, obtaining general information 430, reviewing trends 440, getting medical renewals or refills 450, and getting patient reported outcomes data collection 460, as part of this function the patient quality of life and incentive structure is provided.

One key function is medication tracking, specifically a) the tracking of chelator medications, b) a pharmacy interface that provides a feedback loop for chelation medication prescription renewals; and c) tracking administering and taking medications in order to develop, in part, a plan to enhance a patient's self-care development. With respect to the latter, a drop down menu or information may be provided as to why the medication wasn't taken, so the provider can see trends in barriers, time, or scheduling of medications.

Related to the function of medication tracking is medication renewals or prescription refills. It is important that this contemplated function perform at least one of the following: a) provide an easy way for patient to contact health provider to request timely medication renewal; b) provide incentives/token for taking meds and keeping up renewals which are important for teenager and encourages adherence to medication for chronic illness, c) allows the provider to review renewals, patient progress and to meet with patient; and d) provide a reward framework for the patient.

Another important group of functions for contemplated embodiments comprises lab tracking and trending core clinical indicators for clinical status function. As part of this contemplated embodiment, the following subfunctions are contemplated: a) identification of which ideal tests and numbers would be tracked monthly, quarterly, and annually from clinics, including Thalasemia treatment guidelines, b) tracking changes by the patient system, including the cardiac system, the endocrine system, and/or the liver system; c) providing tracking and trending of serum ferritin monthly, which may be available for download from the clinic health portal or which may include iron loading, ferritin levels, and liver function tests; d) providing tracking of what is normal and abnormal over time, so that patients can visually see results and know test results and tracking; and e) providing considerations for security, HIPPA, and EPIC interfaces.

A contemplated appointment function is very important, including a) ability to have clinic interface with contemplated systems, methods, and apps to help confirm and calendar appointments; b) providing a patient process to sign release forms for HIPPA with clinic; c) providing an interface set with individual clinics with a process for each clinic; d) providing and interacting with a calendar from clinic into app where it will be able to move to other calendar systems; e) allowing the appointment related to the clinic to appear in the app calendar; and f) scheduling blood transfusions.

Contemplated embodiments, as disclosed herein, provide communications functions that are vital. In one embodiment, a contemplated communication function will allow communication with the healthcare provider/nurse practitioner, which would be used for medication renewals/refills, status updates, patient concerns and questions, and a link for oversight of the health providers. Another contemplated communication function is communication with peers, which would be used for sharing experiences with other peers or to provide social interaction that may enhance self care. Yet another contemplated communication function includes providing communication with a support group, which can be used to share experiences, to blog about concerns, care, or questions; or social interaction which may improve self care. Finally, contemplated communication functions may provide general access to the latest research, websites, and resources for Thalassemia patients and their families that is targeted to the specific patent and not “general articles” that can be found on any medical website.

Contemplated embodiments include security functions that may provide unsecured access at the clinic level to provide peer support and access to blogs. The security function may provide moderated security levels that are related to HIPPA requirements or in order to help address patient privacy.

Another key function is patient-reported outcome data collection, so that the patient and the providers can understand the quality of life functions. Each month consider a different dimension—for example, first month of app use have questions from PROM IS tool for physical health. This data input by the patient would be rewarded with incentive. If there are problem areas more questions in the drop down would appear to help provider know what the problem genesis is and this would be flagged to provider. Second month of app use have questions related to emotional health etc. A contemplated method, as described herein, may also work with various other data collection systems in order to gather and analyze the individual patient's data. These additional systems may include an Apple Watch, a FitBit device, another type of smart watch or digital patch, or any other suitable device capable to providing information to the app.

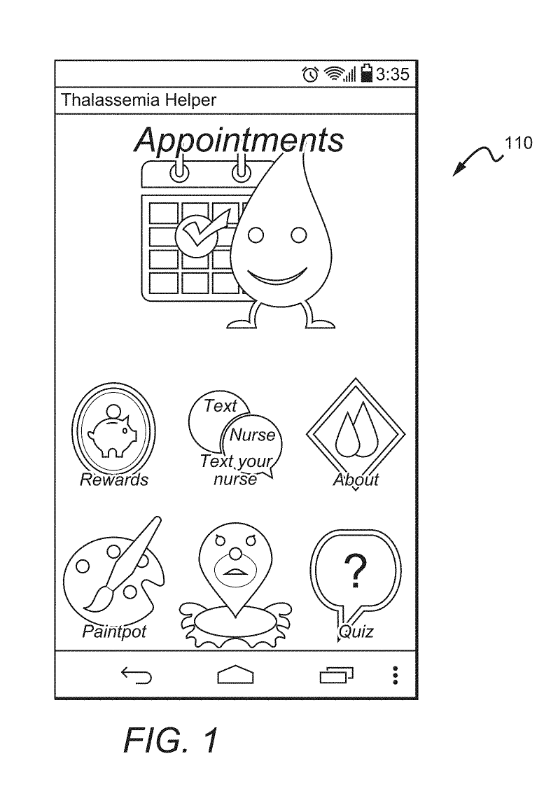

To further explore potential features of a mobile app for children 8-16 years of age with Thalassemia, the nursing faculty and students collaborated with Dr. Youwen Ouyang, a faculty in the CSUSM Computer Science Department, along with several of her students. Through story boarding, the team has identified several key pieces of functionality for a contemplated application, including a calendar function to help patients keeping track of their appointments, and a messaging feature to allow patients to send a text to their provider (FIG. 1: Initial Screen Shot 110 of a contemplated Thalassemia App showing various functionality).

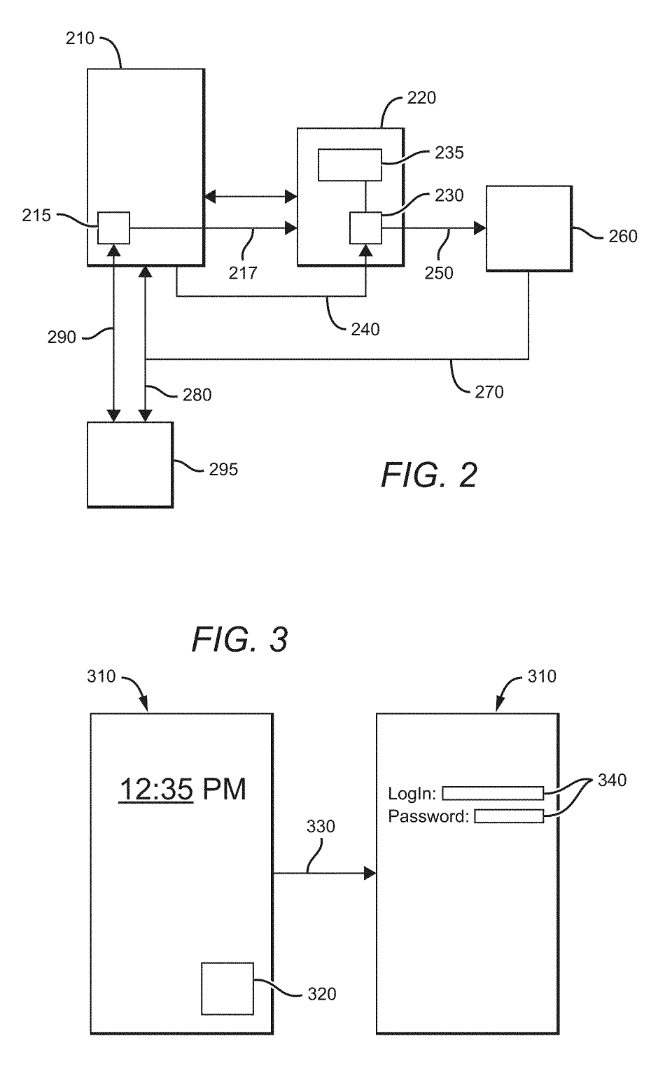

Specifically, as a result of this work systems for providing a chronically ill patient with medical and pharmacological alerts and information were developed that are shown in FIG. 2 and that include: a mobile device 210 comprising a GPS receiver 215, a display, a microprocessor and a wireless communication transceiver, the mobile device programmed to process encrypted medical data, medical tags and medical information through an application; a server 220 comprising a central processing unit 230, a memory 235, a clock and a server communication transceiver that receives input from the wireless communication transceiver of the mobile device, the memory 235 having encrypted medical data, medical tags and medical information stored therein for a plurality of chronically ill patients, the central processing unit 230 programmed to: receive encrypted medical data, medical tags and medical information 240 from the mobile device 210; analyze 250 the encrypted medical data, medical tags and medical information from the mobile device; generate 260 a new set of unique and revised encrypted medical data, medical tags and medical information; provide the new set of unique and revised encrypted medical data, medical tags and medical information to the mobile device without active prompting or direct initiation by the patient, wherein the mobile device updates 270 the application with the unique and revised encrypted medical data, medical tags and medical information, which is then accessible by a user of the mobile device. In some embodiments, the patient is younger than 19 years old. In other embodiments, the patient is younger than 16 years old. In yet other embodiments, the patient is younger than 13 years old.

In other embodiments, a system and method for providing a chronically ill patient with medical alerts, pharmacological alerts and information that includes: a mobile device comprising a GPS receiver 215, a display, a microprocessor and a wireless communication transceiver, the mobile device programmed to process encrypted medical data, medical tags and medical information through an application; a server comprising a central processing unit, a memory, a clock and a server communication transceiver that receives input from the wireless communication transceiver of the mobile device, the memory having encrypted medical data, medical tags and medical information stored therein for a plurality of chronically ill patients, the central processing unit programmed to: receive encrypted medical data, medical tags and medical information from the mobile device; receive GPS information 217 from the wireless communication transceiver from the mobile device; analyze the encrypted medical data, medical tags and medical information from the mobile device; determine 290 location information for a medical facility, healthcare provider or pharmacy 295 utilizing the GPS information from the wireless communication transceiver from the mobile device and the encrypted medical data, medical tags and medical information 280 from the mobile device; and provide the location information to the mobile device, wherein the mobile device notifies a user of the mobile device of this location information—all without active prompting or direct initiation by the patient.

It should be understood that the phrase “without active prompting or direct initiation by the patient” means that the patient is not taking the first step of active initiation, but instead the app is utilizing the data received in an ongoing fashion from the patient to make choices and to initiate information on its own. In some embodiments, the data or medical information is instead being acted on or used to initiate action by the doctors, nurses, or pharmacists who are monitoring the information and/or the patient. It has been mentioned earlier, but this is one of the most important functions this app can perform—in that the patient does not have to be reliable or ready to direct care for himself or herself. This “training” and “monitoring” aspect of this app is a significant key for patients who are chronically ill and are either young or unable to direct care.

Contemplated mobile devices comprise a GPS receiver, a display, a microprocessor and a wireless communication transceiver, the mobile device programmed to process encrypted medical data, medical tags and medical information through an application. It is understood that many contemplated mobile devices also contain Wi-Fi or Bluetooth protocol. Contemplated mobile devices include tablets, smart phones, laptops, wireless masks or glasses, watches, bracelets and any other suitable device.

As mentioned, one of the key components of the current system is encrypted medical data, medical tags, medical information or a combination thereof, which may comprise dosage regimens that are patient-specific and not generalized for the condition or disease, condition or disease-related information that is tailored specifically for the individual patent, based on his or her gender, age, weight, location, IQ and other patient-specific indicators. It should be understood that “medical data, medical tags, and medical information” are considered distinct and individual pieces of information or data. Contemplated information may also include information-based games designed to help children and young people with chronic illnesses or diseases with an understanding of their conditions.

A contemplated server comprises a central processing unit, a memory, a clock and a server communication transceiver that receives input from the wireless communication transceiver of the mobile device. In some embodiments, a contemplated server may be a stand-alone unit or a series of units that are stored in one location. In another contemplated embodiment, a contemplated server may be a system of servers that are located in one facility or in multiple facilities. Contemplated servers may also include Cloud-based functionality, where some of the information isn't stored locally, but instead stored in a virtual server. In some embodiments, contemplated servers are programmed to provide the unique and revised encrypted medical data, medical tags and medical information to a physician, nurse practitioner, health care practitioner, pharmacy or another health care related facility or practitioner.

Contemplated memory comprises or contains encrypted medical data, medical tags and medical information stored therein for a plurality of chronically ill patients, wherein the central processing unit of the server is programmed to: receive encrypted medical data, medical tags and medical information from the mobile device; receive GPS information from the wireless communication transceiver from the mobile device; analyze the encrypted medical data, medical tags and medical information from the mobile device; determine location information for a medical facility, healthcare provider or pharmacy utilizing the GPS information from the wireless communication transceiver from the mobile device and the encrypted medical data, medical tags and medical information from the mobile device; and provide the location information to the mobile device, wherein the mobile device notifies a user of the mobile device of this location information.

A critical component of the contemplated systems is to support patients in the administration of their medication at home. Each child will work with their healthcare providers to set up a customized medication schedule on their device. Such schedule will provide helpful information about the prescribed medications; including dosage, frequency, benefits for adhering to the schedule, warnings for missing dosage, and any special instructions for medication administration. The customized medication schedule will trigger specialized alarms that remind the patient about their medications. Patient's responses to these alarms will be tracked to monitor their medication adherence and be associated with the reward system.

Contemplated systems and applications can also contain functionality that allows the patient to identify clinics and healthcare providers that are in the patient's area when he or she is traveling. This specific functionality is tied to the GPS receiver in the mobile device. In some embodiments, the patient may turn on the GPS receiver in the mobile device and specifically search for information. In some embodiments, if the patient leaves the GPS receiver active in the mobile device, the application may provide this information on an on-going basis without prompting from the patient. This is important, especially if the patient has an emergency and does not have time to walk through turning on the GPS receiver, waiting for the application to locate the patient and provide information.

This functionality is ideal and crucial, in that if the patient has a chronic illness that isn't well known or commonly treated, the patient can identify the closest clinic or doctor who does have experience in this area. Contemplated systems and applications can also contain pharmacy-related functionality, so that if a patient needs a refill of medication, new medication or a change of medication, the healthcare provider can send that information to the app, whereby that information is forwarded to the patient's pharmacy, the nearest pharmacy, if the patient is traveling, and uploaded to the medication functionality on the app, so that the new protocol is implemented and the appropriate reminders set for the patient. It is contemplated that this functionality can proceed without direct prompting or initiating from the patient. This aspect is very important to those who have chronic illnesses and who may not be able to or equipped to be directly responsible for all of the aspects of his or her care at one or multiple points in time. It is important that the team of physicians, nurses, and pharmacists have the information they need when they need it without the patient having to initiate that information transfer.

Contemplated systems comprise a scheduling component, so that healthcare practitioners can calendar follow-up visits or remind the patient to set up an appointment in the future. This functionality is ideal for younger patients, because they don't traditionally schedule their own appointments, but rely solely on their parents or guardians to handle this aspect of their lives.

In order to provide more incentive for children to engage in their own care, the app uses a reward system, such as a coin system, for the providers to award points for patients who keep their appointments. Successful collection of coins unlocks fun games for children such as Paint Bot and Mole Smasher. Another novel feature is the use of quizzes to test the patient's knowledge of his or her condition and to provide information tailored to the child's development and understanding of his or her condition. This feature allows doctors and nurses to provide information in a manner that is fun and engaging for the patient. This feature also allows doctors and nurses to provide updated or new information to the patient, when those resources and information become available in a way that is targeted to the patient.

As the child transitions into pre-teen, teen and young adult stages, contemplated mobile apps can transition with those patients. For example, some of the games and reward systems designed for younger children can be replaced by games and reward systems designed for older children and young adults. In addition, a comprehensive, long-term care protocol can be reliably mapped out and changed as needed for the patient as he or she gets older, grows and matures. Contemplated systems allow the patient and the doctor to be connected more reliably and in a manner that gives both the doctor and the patient flexibility of interaction.

Contemplated systems comprise a password or security component that requires the patient to log-in 340 to the system 320, as shown in FIG. 3. The application 320 on the mobile device 310 will require that the patient update the password or security component on a regular schedule, to lessen the likelihood of compromise of the patient's information. The application will be linked from the health provider-side using security measures in place for patient records and will be updated as needed based on government and practice requirements and best practices.

The use of mobile apps in healthcare for children remains a new and exciting field rich with creative possibilities. Using mHealth apps will foster the goal of providers to facilitate positive healthcare outcomes. By effectively engaging children, healthcare mobile apps hold the potential to enhance lifelong patient self-management, reduce healthcare costs, and improve patients' quality of life.

### REFERENCES

1. Clark, C. C., & Paraska, K. (2013). Health promotion for nurses: A practical guide. Burlington, Mass: Jones & Bartlett Learning.
2. Educative Development Center, & SRI International (2012). EDC/SRI year 2 ready to learn research and evaluation summary report (Report to the CBP PBS ready to learn initiative). Retrieved from http://www-tc.pbskids.org/lab/media/pdfs/research/Y2-EDC-SRI-Year_2_Summary_Report. pdf
3. Eng, D., & Lee, J. (2013). The promise and peril of mobile health applications for diabetes and endocrinology. Pediatric Diabetes, 14(4), 231-238. doi: 10.1111/pedi.12034
4. Kelly, Dr. (2014). Theory to reality: the role of the transition nurse coordinator. British Journal of Nursing. 23(16), 888-894.
5. Fox, S., & Duggan, M. (2012). Mobile health 2012: Half of smartphone owners use their devices to get health information and one-fifth of smartphone owners have health apps (Pew Research Publication). Washington, D.C.: Pew Research Center's Internet & American Life Project. Retrieved on Mar. 2, 2014 from http://pewinternet.org/Reports/2012/Mobile-Health.aspx
6. IMS Institute for Healthcare Informatics. (2013). Patient apps for improved healthcare: from novelty to mainstream (IMS report). Parsipanny, N.J.: Atkins, M., & Gauntlett, C. Retrieved Mar. 1, 2014 from http://www.imshealth.com/deployedfiles/imshealth/Global/Content/Corporate/IMS %20Health%20Institute/Reports/Patient_Apps/IIHI_Patient_Apps_Report. pdf
7. Madden, M., Lenhart, A., Duggan, M., Cortesi, S., & Gasser, U. (2013, Mar. 13). Teens and technology 2013. (Pew Internet Project Publication) Washington, D.C. Retrieved Mar. 5, 2014, from http://www.pewinternet.org/Reports/2012/Teens-and-Tech.aspx
8. Rainie, L., & Fox, S. (2012, May 7). Just-in-time information through mobile connections. (Pew Internet Project Publication) Washington, D.C. Retrieved Mar. 1, 2014, from http://pewinternet.org/Reports/2012/Just-in-time.aspx
9. Reid, S. C., Kauer, S. D., C., D., Khor, A. S., Sanci, L. A., & Patton, G. C. (2013).
10. A mobile phone application for the assessment and management of youth mental health problems in primary care: health service outcomes from a randomized controlled trial of mobile type. BMC Family Practice, 14(84), 1-9. doi:10.1186/1471-2296-14-84

We want to acknowledge and thank Dr. Robert Yamashita, whose life work has been on the study of Thalassemia, for his encouragement in this projects undertaking to serve Thalassemia patients. His astute observations about Thalassemia, healthcare, clinics who serve these patients, and the quality of life of Thalassemia and chronic illness patients has been tremendously appreciated. We would also like to thank The California State University for the University Professional Development funding to support the research and development of the app. Finally, we would like to thank California State University—San Marcos for their support of our work.

Thus, specific embodiments of mobile applications for use with patients who have a chronic illness, including Thalassemia, have been disclosed. It should be apparent, however, to those skilled in the art that many more modifications besides those already described are possible without departing from the inventive concepts herein. The inventive subject matter, therefore, is not to be restricted except in the spirit of the disclosure herein. Moreover, in interpreting the specification, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

TABLE 1
mHealth Apps Currently Available for Children with Chronic Health Conditions

| App Name | Link | Cost | Type of App | Age | Comment |
|---|---|---|---|---|---|
| Carb | http://www.lenny-diabetes.com/carb- | Free | Nutrition/Diabetes | Children and | There are games, a food |
| Counting | counting-with-lenny.html | | Carb counting allows | young adults | guide and quizzes to test |
| with Lenny | | | children with diabetes | | food knowledge |
| US | | | to eat a variety of | | Game looks very bright and |
| | | | foods, just like other | | colorful, using a lion as the |
| | | | children, and increases | | main character. |
| | | | their sense of control | | There is a reward system |
| | | | and confidence in | | where you can win trophies |
| | | | managing their | | |
| | | | diabetes. For parents | | |
| | | | and caregivers, this is | | |
| | | | important in order to | | |
| | | | plan meals and help | | |
| | | | children maintain good | | |
| | | | blood sugar levels. | | |
| Doc | http://www.smartappsforkids.com/2014/01/ | $3.99 | Nutrition/Exercise | 3-7 | Very colorful |
| McStuffins: | review-doc-mcstuffins-promotes- | | | | There are characters which |
| Moving | healthy-living-in-new-disney-app.html | | | | are engaging |
| with Doc | | | | | There are games and a |
| | | | | | popular Disney character. |
| | | | | | Encourages children to eat |
| | | | | | healthy and stay active. |
| | | | | | Strikes a good balance |
| | | | | | between screen time and |
| | | | | | promoting activities that will |
| | | | | | make children ages 3-7 get |
| | | | | | off the couch. |
| Kids | http://www.imedicalapps.com/2013/03/kids- | Free | Asthma Management | Any child or | Media and illustrations are |
| Beating | beating-asthma-app-children-learn-asthma/ | | This app tries to create | teenager who | made to target children |
| Asthma | | | a way for children to | has asthma | The app is not currently |
| | | | learn about asthma. | | complete. |
| | | | | | The information may go |
| | | | | | beyond the level of |
| | | | | | understanding that children |
| | | | | | under 12 years old need to |
| | | | | | have |
| iAsthma in | https://itunes.apple.com/us/app/iasthma- | $.0.99 | Asthma Management | Children | Pediatricians - recommend |
| Control | in-control/id329847125?mt=8 | | Teaches children how | | this App |
| | | | to care and manage | | Has apt log, teaching and |
| | | | their asthma | | quizzes |
| Asthma | http://www.nationalasthma.org.au/asthma-tools/ | Free | Asthma Management | Children | Reminds day-to-day |
| buddy | asthma-action-plans/asthmabuddy | | | | asthma medications. |
| | | | | | Uses large pictures similar |
| | | | | | to the children pain |
| | | | | | assessment tool w/ |
| | | | | | different faces. Could be |
| | | | | | more colorful |
| Monster | http://www.sanofi.co.uk/l/gb/en/ | Free | Type I Diabetes | 6-13 | Engages children in their |
| Manor App | layout.jsp?cnt=BCC9BE08- | | Management | | health management and |
| | 91D8-4C1E-8D98-666907A88192 | | Helps families of | | improve adherence to |
| | | | young children with | | treatment while having fun. |
| | | | Type 1 Diabetes | | Very colorful and looks fun. |
| | | | manage their blood | | Games where they can |
| | | | sugar | | unlock monsters. |
| mySugr | https://mysugr.com/junior/ | Free | Diabetes | Children | Very colorful and simple to |
| Junior | | | Management | | use. Like the use of |
| | | | Provides | | symbols instead of a bunch |
| | | | communication | | of words |
| | | | between children living | | Enables children to be |
| | | | with diabetes and their | | more independent. |
| | | | caregivers. | | |
| Sugarpoint | http://www.iternum.com/apps/sugarpointkids/ | $3.99 | Diabetes | Children | Not very fun looking or |
| Kids | | | Management | | engaging. |
| | | | Supports parents and | | Just allows you to track |
| | | | children in their daily | | your glucose readings, |
| | | | diabetes management | | exercise, BP, meals, but |
| | | | | | doesn't have any games, or |
| | | | | | anything that would seem |
| | | | | | fun for a child. |
| Motionmaze | https://itunes.apple.com/us/app/motionmaze/ | Free | Exercise | Children | It's a puzzle game that |
| | id463896292?mt=8 | | | | requires physical |
| | | | | | movement to play. |
| Eat & | http://mediaproductions.nmsu.edu/eatmove/ | Free | Exercise and | Children | App looks great for |
| Move-O- | | | Nutrition | | children, very colorful and |
| Matic | | | Encourages young | | interactive |
| | | | people to develop and | | |
| | | | maintain healthy, | | |
| | | | active lifestyles. | | |
| iLearnWith | http://www.ilearnwith.com/our-apps/nutrition/ | Free to try | Nutrition and Healthy | Children | Over 5 million downloads |
| | | and then | Eating | | “Brain Toy Award” - |
| | | buy, then | | | Academics' Choice Awards |
| | | $2.99-$19.99 | | | 2012 |
| | | | | | The app looks great and |
| | | | | | interactive. Very colorful |
| | | | | | and great use of characters |
| Smash | http://smashyourfood.foodnme.com | $0.99 | Nutrition | Children | Winner in Michelle |
| Your Food | | | Motivates you to make | | Obama's National contest |
| | | | healthier food choices. | | at The White House |
| | | | | | App looks fun and children |
| | | | | | love to smash stuff [symbol] |
| Wellapets | http://www.wellapets.com | $2.99 | Asthma | Children | You adopt a pet and |
| | | | Teach children about | | manage their asthma, by |
| | | | asthma management | | doing so it teaches the |
| | | | | | child about their asthma. |
| | | | | | Very interactive and |
| | | | | | educative |
| | | | | | Improve children' |
| | | | | | knowledge and self-efficacy |
| | | | | | regarding their health |
| | | | | | Help children remember to |
| | | | | | take their medication at the |
| | | | | | right times |
| | | | | | Keep children' health |
| | | | | | conditions under control |
| | | | | | better |

TABLE 2
Links for Website Reviews of mHealth Apps

| Website | Link |
|---|---|
| 3 Diabetes Games that will | http://timesulin.com/theblog/3-diabetes-games-will-educate-kids-diabetes/ |
| Educate Children About | |
| Diabetes | |
| Mobile Asthma Apps | http://www.medscape.com/viewarticle/806941_5 |
| Top-Rated Diabetes Apps | http://diabetes.ufl.edu/my-diabetes/diabetes-resources/diabetes-apps/ |

We claim:

1. A method for providing a chronically ill patient with medical alerts, pharmacological alerts and information, comprising:
providing a patient with a chronic illness;
providing a mobile device comprising a GPS receiver, a display, a microprocessor and a wireless communication transceiver, the mobile device programmed to process encrypted medical data, medical tags and medical information through an application;
providing a server comprising a central processing unit, a memory, a clock and a server communication transceiver that receives input from the wireless communication transceiver of the mobile device, the memory having encrypted medical data, medical tags and medical information stored therein for a plurality of chronically ill patients, the central processing unit programmed to implement the following method comprising:
receiving the patient's encrypted medical data, medical tags and medical information from the mobile device;
analyzing the encrypted medical data, medical tags and medical information from the mobile device;
generating a new set of the patient's unique and revised encrypted medical data, medical tags and medical information;
providing the new set of unique and revised encrypted medical data, medical tags and medical information to the mobile device, wherein the mobile device updates the application with the unique and revised encrypted medical data, medical tags and medical information, which is then accessible by a user of the mobile device.

2. The method of providing a chronically ill patient with medical alerts, pharmacological alerts and information of claim 1, further comprising the server programmed to provide the unique and revised encrypted medical data, medical tags and medical information to a physician.

3. The method of providing a chronically ill patient with medical alerts, pharmacological alerts and information of claim 1, wherein the illness is Thalassemia.

4. The method of providing a chronically ill patient with medical alerts, pharmacological alerts and information of claim 1, wherein providing the new set of unique and revised encrypted medical data, medical tags and medical information to the mobile device is initiated or completed without prompting from the patient.

5. The method for providing a chronically ill patient with medical alerts, pharmacological alerts and information of claim 1, further comprising a secure log in screen on the mobile device when the system is initiated.

6. The method for providing a chronically ill patient with medical alerts, pharmacological alerts and information of claim 1, further comprising at least one game.

7. The method for providing a chronically ill patient with medical alerts, pharmacological alerts and information of claim 1, further comprising at least one reward option.

8. The method for providing a chronically ill patient with medical alerts, pharmacological alerts and information of claim 1, further comprising at least one function that allows direct contact with a health care practitioner.

9. A method for providing a chronically ill patient with medical alerts, pharmacological alerts and information, comprising:
providing a chronically ill patient;
providing a mobile device comprising a GPS receiver, a display, a microprocessor and a wireless communication transceiver, the mobile device programmed to process encrypted medical data, medical tags and medical information through an application;
providing a server comprising a central processing unit, a memory, a clock and a server communication transceiver that receives input from the wireless communication transceiver of the mobile device, the memory having encrypted medical data, medical tags and medical information stored therein for a plurality of chronically ill patients, the central processing unit programmed to the central processing unit programmed to implement the following method comprising:
receiving the patient's encrypted medical data, medical tags and medical information from the mobile device;
receiving GPS information from the wireless communication transceiver from the mobile device;
analyzing the encrypted medical data, medical tags and medical information from the mobile device;
determining location information for a medical facility, healthcare provider or pharmacy utilizing the GPS information from the wireless communication transceiver from the mobile device and the encrypted medical data, medical tags and medical information from the mobile device;
providing the location information to the mobile device without prompting from the patient, wherein the mobile device notifies a user of the mobile device of this location information.

10. The method for providing a chronically ill patient with medical alerts, pharmacological alerts and information of claim 9, further comprising the server programmed to provide the unique and revised encrypted medical data, medical tags and medical information to the medical facility, healthcare provider or pharmacy.

11. The method for providing a chronically ill patient with medical alerts, pharmacological alerts and information of claim 9, further comprising a secure log in screen on the mobile device when the system is initiated.

12. The method for providing a chronically ill patient with medical alerts, pharmacological alerts and information of claim 9, further comprising at least one game.

13. The method for providing a chronically ill patient with medical alerts, pharmacological alerts and information of claim 9, further comprising at least one reward option.

14. The method for providing a chronically ill patient with medical alerts, pharmacological alerts and information of claim 9, further comprising at least one function that allows direct contact with a health care practitioner.

15. A method for providing a chronically ill patient with medical alerts, pharmacological alerts and information, comprising:
providing a patient with a chronic illness;
providing a mobile device comprising a GPS receiver, a display, a microprocessor and a wireless communication transceiver, the mobile device programmed to process encrypted medical data, medical tags and medical information through an application;
providing a server comprising a central processing unit, a memory, a clock and a server communication transceiver that receives input from the wireless communication transceiver of the mobile device, the memory having encrypted medical data, medical tags and medical information stored therein for a plurality of chronically ill patients, the central processing unit programmed to implement the following method comprising:
receiving the patient's specific encrypted medical data, medical tags and medical information from the mobile device;
analyzing the encrypted medical data, medical tags and medical information from the mobile device;
generating a new set of the patient's unique and revised encrypted medical data, medical tags and medical information;
providing the new set of unique and revised encrypted medical data, medical tags and medical information to the mobile device wherein providing the new set of unique and revised encrypted medical data, medical tags and medical information to the mobile device is initiated or completed without prompting from the patient.

16. The method for providing a chronically ill patient with medical alerts, pharmacological alerts and information of claim 15, wherein the chronic illness is Thalassemia.

17. The method for providing a chronically ill patient with medical alerts, pharmacological alerts and information of claim 15, wherein the chronic illness is Thalassemia and the patient is younger than 19 years old.
